(19) United States
(12) Patent Application Publication
(10) Pub. No.: US 2004/0166178 A1
(43) Pub. Date: Aug. 26, 2004

(54) 3-O-acetyl-11-ketoboswellic acid for relaxing the skin

(75) Inventors: Alain Meybeck, Courbevoie (FR); Alain Zanvit, Paris (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 10/737,897

(22) Filed: Dec. 18, 2003

(30) Foreign Application Priority Data
Feb. 3, 2003 (FR) 0301205

(51) Int. Cl. A61K 35/78

(52) U.S. Cl. 424/725; 424/748

(57) ABSTRACT

The present invention relates to the use of 3-O-acetyl-11-ketoboswellic acid, or of a plant extract or composition containing it, for topical application to the skin, as an agent to soften lines and/or relax the skin. The plant extract is in particular an extract of Boswellia serrata.

### REFERENCE TO PRIOR APPLICATIONS

This application claims priority to French patent application 0301205 filed Feb. 3, 2003, incorporated herein by reference.

### FIELD OF THE INVENTION

The present invention relates to the use of 3-O-acetyl-11-ketoboswellic acid, or of a composition, plant extract, etc. comprising it, to soften lines and/or relax the skin. Preferred embodiments of the invention include the use of a composition suitable for topical application to the skin comprising 3-O-acetyl-11-ketoboswellic acid.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Women, and even men, currently have a tendency to want to appear younger for as long as possible, and so they seek to tone down signs of ageing of the skin, primarily wrinkles and fine lines. In this respect, advertisements and fashion promote products-intended to keep the skin radiant and wrinkle-free, the trade marks of a young skin, for as long as possible, all the more so since physical appearance has an effect on the psyche and/or morale.

Until now, wrinkles and fine lines have been treated using cosmetic products containing active agents which have an effect on the skin, for example by moisturizing it or by improving cell renewal, or else by promoting the synthesis of collagen from which cutaneous tissue is formed, or by preventing its degradation.

Although these treatments make it possible to have an effect on wrinkles and fine lines due to chronological or intrinsic ageing, and also on those due to photoageing, they do not have any effect on expression lines.

Expression lines are in fact produced by mechanisms different from those generating lines due to ageing.

Precisely, they are produced by the stress exerted on the skin by the facial muscles which produce facial expressions. Depending on the shape of the face, the frequency of expressions and the possible existence of any tics, they can appear in childhood. Age, along with certain environmental factors such as exposure to the sun, do not have any affect on their genesis but can make them deeper and-render them permanent.

Expression lines are characterized by the presence of furrows at the periphery of the orifices, namely the nose (nasogenic furrows), the mouth (para-buccal lines and bitterness lines) and the eyes (crows feet), around which the facial muscles are located, and also between the eyebrows (glabellar lines or frown lines) and on the forehead.

Until now, the only means commonly used for dealing with expression lines are, firstly, botulinum toxin, which is in particular injected into the glabellar lines (see J. D. Carruthers et al., J. Dermatol. Surg. Oncol., 1992, 18, pp. 17-21) and, secondly, degradable collagen-based, hyaluronic acid-based or polylactic acid-based implants.

In addition, as an alternative to these medical techniques requiring the services of a practitioner, the assignee has proposed various compounds capable of providing a muscle-relaxing effect when they are applied topically to the skin, thus making it possible to act on expression lines in a different manner. Among these compounds, mention may in particular be made of antagonists for calcium channel-associated receptors (FR-2 793 681), and in particular manganese and its salts (FR-2 809 005) and alverine (FR-2 798 590); and agonists for chloride channel-associated receptors, including glycine (EP-0 704 210) and certain extracts of Iris pallida (FR-2 746 641).

However, there is still a need for effective compounds for smoothing or toning down expression lines by relaxing the skin or loosening facial muscles.

### DETAILED DESCRIPTION OF THE PREFERRED EMBODIMENTS

Now, the inventors have surprisingly discovered that 3-O-acetyl-11-ketoboswellic acid makes it possible to satisfy this need.

This compound is a well known constituent of various plants, in particular of the genus Boswellia and even more particularly of the species Boswellia serrata.

Boswellia serrata is a tree of Indian origin which produces a resinous exudate when its bark is cut. This exudate contains polysaccharides, aromatic residues and resin gums, but especially penta- and tetracyclic terpenic acids, including boswellic acid and also derivatives thereof acetylated in the 3-position and/or carbonylated in the 11-position (Pardhy & Bhattacharyya, Ind. J. Chem., 16B: 176-178, 1978). These compounds are commonly referred to as “boswellic acids” in the literature.

Boswellic acids are known for their anti-inflammatory properties due to their ability to inhibit leukotriene synthesis by inhibiting 5-lipoxygenase [(SINGH G. B. et al., Anti-inflammatory actions of boswellic acids, Phytomedicine 3(1): 81-85 (1996), SINGH G. B. et al., Pharmacology of an extract of salai guggal ex-Boswellia serrata, a new non-steroidal anti-inflammatory agent, Agents and actions 18(3-4): 407-412 (1986), SAFAYHI H. et al., Boswellic acids: novel specific nonredox inhibitors of 5-lipoxygenase, J. Pharmacol, Exp. Ther. 261, 1143-1146 (1992)].

The commercially available extracts of Boswellia serrata are thus recommended against skin irritations, in particular in hair-removing products, and sunburn, in particular in aftersun creams.

Other properties have been attributed to boswellic acids, and in particular an ability to inhibit human leukocyte elastase activity [SAFAYHI H. et al., Inhibition by boswellic acids of human leukocyte elastase, J. Pharmacol. Exp. Ther. 281: 460-463 (1997)], which has led to their use being envisaged in the treatment of arthritis (EP-0 854 709). It has also been demonstrated that they inhibit some proteases involved in skin desquamation (WO 01/74327) and collagenases (JP2000-154 131). It has thus been advised to use Boswellia serrata extracts in topical cosmetic compositions intended for the treatment of dry or aged skin, in particular for preventing the formation of wrinkles and fine lines.

However, it has never yet been suggested, to the inventor's knowledge, that boswellic acids, and more particularly 3-O-acetyl-11-ketoboswellic acid, can exhibit properties that justify their use for relaxing the skin. A subject of the present invention is therefore the use of 3-O-acetyl-11-ketoboswellic acid, alone or in a composition suitable for topical application to the skin, as an agent for softening lines and/or relaxing the skin. Such use is preferably a use by a person desirous of softening lines and/or relaxing the skin.

The 3-O-acetyl-11-ketoboswellic acid may be used as, or be added to or present in the invention compositions, in the form of a plant extract. Preferred plant extracts containing 3-O-acetyl-11-ketoboswellic acid include extracts of plants of the genus Commiphora and of the genus Boswellia, the latter being preferred for use in the present invention.

As plant extracts from the genus Commiphora, mention may be made of the following species: Commiphora myrrha, Commiphora mukul, Commiphora opobalsamum, Commiphora playfairii, Commiphora abyssincia and Commiphora simplicifolia, the variety Commiphora mukul being preferred.

As plant extracts from the genus Boswellia, mention may be made of the following species: Boswellia serrata, Boswellia carteri, Boswellia papyrifera, Boswellia frereana, Boswellia thurifera, Boswellia glabra, Boswellia oblongata and Boswellia socotrana.

An extract of Boswellia serrata is preferably used to implement the present invention.

Extracts of Boswellia serrata which have been assayed for 3-O-acetyl-11-ketoboswellic acid content are commercially available from: the company QUEST under the commercial name Soothex®, the company SABINSA under the commercial name Boswellin®, the company ZANDU under the commercial name R3® and the company NATURACTIVA under the commercial name ViaPure Boswellia®.

These extracts contain between 0.3 and 3.0% of 3-O-acetyl-11-ketoboswellic acid, relative to the total weight of the extract.

The plant extract used according to the invention may, as a variant, be obtained by extraction of resinous exudate of Boswellia serrata using alcoholic solvents such as ethanol or isopropanol, to obtain an alcoholic extract, optionally followed by alkali treatment of the alcoholic extract, intended to isolate the acid fraction, and then extraction of the alkali-treated alcoholic extract using an organic solvent, such as ethyl acetate, to produce an aqueous phase and an organic phase, and optionally acid treatment of the aqueous phase thus obtained in order to precipitate the active acid fraction of the extract.

The 3-O-acetyl-11-ketoboswellic acid can be isolated from the preceding active fraction by known chromatographic techniques, as described in particular in application EP-0 755 940, or else according to the process described in application WO 02/085921, or it can be obtained commercially from the company CHROMADEX under the commercial reference 02570.

The composition according to the invention is intended in a preferred embodiment to be applied to the areas of the face or of the forehead which have expression lines, and/or on individuals exhibiting expression lines.

It is thus preferably applied to nasogenic furrows, lines between the eyebrows and lines on the forehead. Preferably, the composition is applied by massaging into the forehead.

The amount of 3-O-acetyl-11-ketoboswellic acid which can be used according to the invention depends of course on the desired effect and can therefore vary within a wide range. In this regard the amount is preferably a result effective amount, easily determined by one of ordinary skill in the art in view of this disclosure. To give an order of magnitude, the composition used according to the invention may comprise from 0.001 to 1% by weight, preferably from 0.001 to 0.1% by weight, of 3-O-acetyl-11-ketoboswellic acid, relative to the total weight of the composition. If a plant source of this compound is used, the amount of plant extract used is adjusted to take into account the amount of boswellic acid derivative in the extract.

The composition according to the invention is preferably suitable for topical application to the skin and it therefore contains a physiologically acceptable medium, i.e. a medium which is compatible with the skin.

This composition may be more or less fluid and have the appearance of a white or coloured cream, of an ointment, of a milk, of a lotion, of a serum, of a paste or of a foam. It may also be in solid form, in particular in the form of a stick. It may be used as a care product and/or as a make-up product for the skin.

The composition according to the invention may be in any form, including all the pharmaceutical forms normally used in the cosmetics field, and it may in particular be in the form of an optionally gelled oily solution, of a dispersion, optionally two-phase, of the lotion type, of an emulsion obtained by dispersion of a fatty phase in an aqueous phase (O/W) or vice versa (W/O), or of a triple emulsion (W/O/W or O/W/O) or of a vesicular dispersion of the ionic and/or nonionic type. These compositions are prepared according to the usual methods. According to this invention, a composition in the form of an oil-in-water emulsion is preferably used.

When the composition used according to the invention is an emulsion, the proportion of the fatty phase can range from 5 to 80% by weight, and preferably from 5 to 50% by weight, relative to the total weight of the composition. The oils, the emulsifiers and the coemulsifiers used in the composition in the form of an emulsion are chosen from those conventionally used in the field under consideration. The emulsifier and the coemulsifier are present in the composition in a proportion ranging from 0.3 to 30% by weight, and preferably from 0.5 to 20% by weight, relative to the total weight of the composition.

As oils which can be used in the invention, mention may be made of hydrocarbons of mineral origin (mineral oil) or synthetic origin (liquid petroleum jelly, isohexadecane), oils of plant origin (apricot kernel oil, liquid fraction of karite butter, avocado oil, soybean oil), oils of animal origin (lanolin), synthetic oils (perhydrosqualene, pentaerythrityl tetraoctanoate), silicone oils (cyclopentasiloxane and cyclohexasiloxane) and fluoro oils (perfluoropoly-ethers). Fatty substances that may also be used include fatty alcohols (cetyl alcohol or stearyl alcohol), fatty acids (stearic acid) and waxes (carnauba wax, ozokerite, beeswax).

As emulsifiers and coemulsifiers which can be used in the invention, mention may be made, for example, of fatty acid esters of polyethylene glycol, such as PEG-100 stearate and PEG-20 stearate, and fatty acid esters of glycerol, such as glyceryl stearate.

In a known manner, the composition used according to the invention may also contain adjuvants that are common in the cosmetics field, such as hydrophilic or lipophilic gelling agents, hydrophilic or lipophilic active agents, preserving agents, antioxidants, solvents, fragrances, fillers, screening agents, pigments, odour absorbers and dyestuffs. The amounts of these various adjuvants are those conventionally used in the field under consideration, for example from 0.01 to 20% of the total weight of the composition. Depending on their nature, these adjuvants may be introduced into the fatty phase, into the aqueous phase or into the lipid vesicles. In any event, these adjuvants, and also the proportions thereof, will preferably be chosen so as not to harm the desired properties of the 3-O-acetyl-11-ketoboswellic acid.

As hydrophilic gelling agents, mention may in particular be made of carboxyvinyl polymers (carbomer), acrylic copolymers such as acrylate/alkyl acrylate copolymers, polyacrylamides, polysaccharides, natural gums and clays, and, as lipophilic gelling agents, mention may be made of modified clays, for instance bentones, metal salts of fatty acids, hydrophobic silica and polyethylenes.

As fillers, mention may, for example, be made of polyamide (Nylon) particles in spherical form of in the form of microfibres; polymethyl methacrylate microspheres; ethylene-acrylate copolymer powders; expanded powders such as hollow microspheres and in particular microspheres formed from a terpolymer of viriylidene chloride, of acrylonitrile and of methacrylate and sold under the name EXPANCEL by the company Kemanord Plast; powders of natural organic materials such as starch powders, in particular corn starch, wheat starch or rice starch, which may or may not be crosslinked, such as the starch powders crosslinked with octenylsuccinate anhydride; silicone resin microbeads such as those sold under the name TOSPEARL by the company Toshiba Silicone; silica; metal oxides, such as titanium dioxide or zinc oxide; mica; and mixtures thereof.

As active agents, it will be advantageous to introduce into the composition used according to the invention at least one compound chosen from: desquamating agents; moisturizers, depigmenting agents; anti-glycation agents; NO-synthase inhibitors; agents for stimulating the synthesis of dermal or epidermal macromolecules and/or for preventing their degradation; agents for stimulating fibroblast and/or keratinocyte proliferation or for stimulating keratinocyte differentiation; muscle-relaxing or dermo-relaxing agents; tensioning agents; antipollution agents and/or free-radical scavengers; and mixtures thereof.

Examples of such additional compounds are given below.

1. Desquamating Agents

The term “desquamating agents” means any compound capable of acting:

either directly on the desquamation by promoting exfoliation, such as β-hydroxy acids, in particular salicylic acid and its derivatives (including 5-n-octanoylsalicylic acid); α-hydroxy acids, such as glycolic acid, citric acid, lactic acid, tartaric acid, malic acid or mandelic acid; urea; gentisic acid; oligofucoses; cinnamic acid; extract of Saphora japonica; resveratrol;

or on the enzymes involved in desquamation or degradation of corneodesmosomes, glycosidases, stratum corneum chymotryptic enzyme (SCCE), or even other proteases (trypsin, chymotrypsin-like). Mention may be made of agents for chelating mineral salts: EDTA; N-acyl-N,N′,N′-ethylenediaminetriacetic acid; amino-sulphonic compounds and in particular (N-2-hydroxy-ethylpiperazine-N-2-ethane)sulphonic acid (HEPES); derivatives of 2-oxothiazolidine-4-carboxylic acid (procysteine); derivatives of alpha-amino acids of the glycine type (as described in EP-0 852 949, and also sodium methylglycinediacetate sold by BASF under the commercial name TRILON M); honey; sugar derivatives such as O-octanoyl-6-D-maltose and N-acetylglucosamine.

2. Moisturizer

The term “moisturizer” means:

either a compound which acts on the barrier function in order to maintain the moisturization of the stratum corneum, or an occlusive compound. Mention may be made of ceramides, sphingoid-based compounds, lecithins, glycosphingolipids, phospholipids, cholesterol and its derivatives, phytosterols (stigmasterol, β-sitosterol or campesterol), essential fatty acids, 1,-2-diacyl-glycerol, 4-chromanone, pentacyclic triterpenes such as ursolic acid, petroleum jelly and lanolin;

or a compound which directly increases the water content of the stratum corneum, such as threalose and its derivatives, hyaluronic acid and its derivatives, glycerol, pentanediol, sodium pidolate, serine, xylitol, sodium lactate, polyglyceryl acrylate, ectoin and its derivatives, chitosan, oligosaccharides and polysaccharides, cyclic carbonates, N-lauroyl-pyrrolidonecarboxylic acid and N-α-benzoyl-L-arginine;

or a compound which activates the sebaceous glands, such as DHEA and its 7-oxide and/or 17-alkyl derivatives, sapogenins, and vitamin D and its derivatives.

3. Depigmenting Agent

The depigmenting agents which can be incorporated into the composition according to the present invention comprise, for example, the following compounds: kojic acid; ellagic acid; arbutin and its derivatives such as those described in patent applications EP-895 779 and EP-524 109; hydroquinone; aminophenol derivatives such as those described in patent applications WO 99/10318 and WO 99/32077, and in particular N-cholesteryloxycarbonyl-para-aminophenol and N-ethyloxycarbonyl-para-aminophenol; iminophenol derivatives, in particular those described in patent application WO 99/22707; L-2-oxothiazolidine-4-carboxylic acid or procysteine, and also its salts and esters; ascorbic acid and its derivatives, in particular ascorbyl glucoside; and plant extracts, in particular extracts of liquorice, of mulberry and of skullcap, without this list being limiting.

4. Anti-Glycation Agent

The term “anti-glycation agent” means a compound for preventing and/or reducing the glycation of skin proteins, in particular of dermal proteins such as collagen.

Examples of anti-glycation agents are plant extracts of the Ericaceae family, such as an extract of blueberry (Vaccinium angustifolium); ergothioneine and its derivatives; and hydroxystilbenes and their derivatives, such as resveratrol and 3,3′,5,5′-tetra-hydroxystilbene.

5. NO-Synthase Inhibitor

Examples of NO-synthase inhibitors which are suitable for use in the present invention comprise in particular an extract of a plant of the species Vitis vinifera which is in particular sold by the company Euromed under the name Leucocyanidines de raisins extra, or else by the company Indena under the name Leucoselect®, or finally by the company Hansen under the name Extrait de marc de raisin; an extract of a plant of the species Olea europaea which is preferably obtained from olive tree leaves and is in particular sold by the company VINYALS in the form of a dry extract, or by the company Biologia & Technologia under the commercial name Eurol BT; and an extract of a plant of the species Gingko biloba which is preferably a dry aqueous extract of this plant, sold by the company Beaufour under the commercial name Ginkgo biloba extrait standard.

6. Agent for Stimulating the Synthesis of Dermal or Epidermal Macromolecules and/or for Preventing their Degradation

Among the active agents for stimulating dermal macromolecules or for preventing their degradation, mention may be made of those which act:

either on collagen synthesis, such as extracts of Centella asiatica; asiaticosides and derivatives; ascorbic acid or vitamin C and its derivatives; synthetic peptides such as iamin, biopeptide CL or the palmitoyloligopeptide sold by the company SEDERMA; peptides extracted from plants, such as the soybean hydrolysate sold by the company COLETICA under the commercial name Phytokine®; and plant hormones such as auxins and lignans;

or on elastin synthesis, such as the extract of Saccharomyces cerivisiae sold by the company LSN under the commercial name Cytovitin®; and the extract of the alga Macrocystis pyrifera sold by the company SECMA under the commercial name Kelpadelie®;

or on glycosaminoglycan synthesis, such as the product of milk fermentation by lactobacillus vulgaris, sold by the company BROOKS under the commercial name Biomin yogourth®; the extract of the brown alga Padina pavonica sold by the company ALBAN MÜLLER under the commercial name HSP3®; and the extract of Saccharomyces cerevisiae available in particular from the company SILAB under the commercial name Firmalift® or from the company LSN under the commercial name Cytovitin®;

or on fibronectin synthesis, such as the extract of the zooplankton Salina sold by the company SEPORGA under the commercial name GP4G®; the yeast extract available in particular from the company ALBAN MÜLLER under the commercial name Drieline®; and the palmitoyl pentapeptide sold by the company SEDERMA under the commercial name Matrixil®;

or on the inhibition of metalloproteinases (MMPs), such as more particularly MMP 1, 2, 3 or 9. Mention may. be made of: retinoids and derivatives, oligopeptides and lipopeptides, lipoamino acids, the malt extract sold by the company COLETICA under the commercial name Collalift®; extracts of blueberry or of rosemary; lycopene; isoflavones, their derivatives or the plant extracts containing them, in particular soybean extracts (sold for example by the company ICHIMARU. PHARCOS under the commercial name Flavosterone SB®), red clover extracts, flax extracts, kakkon extracts, or sage extracts;

or on the inhibition of serine proteases such as leukocyte elastase or cathepsin G. Mention may be made of: the peptide extract of Leguminosa (Pisum sativum) seeds sold by the company LSN under the commercial name Parelastyl ; heparinoids; and pseudodipeptides such as {2-[acetyl-(3-trifluoromethylphenyl)amino]-3-methyl-butyrylamino}acetic acid.

Among the active agents which stimulate epidermal macromolecules, such as fillagrin and keratins, mention may in particular be made of the extract of lupin sold by the company SILAB under the. commercial name Structurine®; the extract of Fagus sylvatica beech buds sold by the company GATTEFOSSE under the trade name Gatuline®; and the extract of the zooplankton Salina sold by the company SEPORGA under the trade name GP4G®.

7. Agent for Stimulating Fibroblast or Keratinocyte Proliferation and/or Keratinocyte Differentiation

The agents for stimulating fibroblast proliferation which can be used in the composition according to the invention may, for example, be chosen from plant proteins or polypeptides, in particular extracted from soybean (for example a soybean extract sold by the company LSN under the name Eleseryl SH-VEG 8® or sold by the company SILAB under the commercial name Raffermine®); and plant hormones such as giberrellins and cytokines.

The agents for stimulating keratinocyte proliferation which can be used in the composition according to the invention comprise in particular retinoids such as retinol and its esters, including retinyl palmitate; phloroglucinol; the extracts of nut cakes sold by the company GATTEFOSSE; and the extracts of Solanum tuberosum sold by the company SEDERMA.

The agents for stimulating keratinocyte differentiation comprise, for example, minerals such as calcium; the extract of lupin sold by the company SILAB under the commercial name Photopreventine®; sodium beta-sitosteryl sulphate sold by the company SEPORGA under the commercial name Phytocohesine®; and the extract of corn sold by the company SOLABIA under the commercial name Phytovityl ; and.lignans such as secoisolariciresinol.

8. Muscle-Relaxing or Dermo-Relaxing Agent

The composition according to the invention may comprise other muscle-relaxing or dermo-relaxing agents, among which mention may.in particular be made of alverine and its salts, in particular alverine citrate, sapogenins such as diosgenin and the natural extracts containing it (such as extracts of wild yam), certain carbonylated secondary and tertiary amines, organic or inorganic salts of metals, especially of manganese, in particular manganese gluconate, adenosine, and also the hexapeptide argireline R sold by the company LIPOTEC. Mention may also be made of certain fragrancing compositions having a dermo-relaxing effect.

9. Tensioning Agent

The term “tensioning agent” means a compound capable of exerting tension on the skin, the effect of which is to temporarily tone down irregularities on the skin's surface, such as wrinkles and fine lines.

Among the tensioning agents which can be used in the composition according to the present invention, mention may be made in particular of:

(1) synthetic polymers, such as polyurethane latices or acrylic-silicone latices, in particular those described in patent application EP-1038519, such as a propyl-thio(polymethyl acrylate), propylthio(polymethyl methacrylate) and propylthio(polymethacrylic acid) grafted polydimethylsiloxane, or alternatively a propylthio(polyisobutyl methacrylate) and propylthio-(polymethacrylic acid) grafted polydimethylsiloxane. Such grafted silicone polymers are in particular sold by the company 3M under the commercial names VS 80, VS 70 or LO21,

(2) polymers of natural origin, in particular (a) polyholosides, for example (i) in the form of starch derived in particular from rice, from corn, from potato, from cassava, from pea, from Triticum aestivum wheat, from oat, etc., or (ii) in the form of carrageenans, alginates, agars, gellans, cellulose-based polymers and pectins, advantageously in aqueous dispersion of gel microparticles, and (b) latices consisting of shellac resin, sandarac gum, dammar resins, elemi gums, copal resins and cellulose derivatives, and mixtures thereof,

(3) plant proteins and protein hydrolysates, in particular from corn, from rye, from Triticum aestivum wheat, from buckwheat, from sesame, from spelt, from pea, from bean, from lentil, from soybean, and from lupin,

(4) mixed silicates, especially phyllosilicates and in particular Laponites,

(5) wax microparticles chosen, for example, from carnauba wax, candelilla wax and alfalfa wax,

(6) colloidal particles of inorganic filler with a number-average diameter of between 0.1 and 100 nm, preferably between 3 and 30 nm, chosen, for example, from: silica, silica-alumina composites, cerium oxide, zirconium oxide, alumina, calcium carbonate, barium sulphate, calcium sulphate, zinc oxide and titanium dioxide.

10. Antipollution Agent or Free-Radical Scavenger

The term “antipollution agent” means any compound capable of trapping ozone, monocyclic or polycyclic aromatic compounds such as benzopyrene and/or heavy metals such as cobalt, mercury, cadmium and/or nickel. The term “free-radical scavenger” means any compound capable of trapping free radicals.

As ozone-trapping agents which can be used in the composition according to the invention, mention may in particular be made of vitamin C and its derivatives, including ascorbyl glucoside; phenols and polyphenols, in particular tannins, ellagic acid and tannic acid; epigallocatechin and natural extracts containing it; extracts of olive tree leaf; extracts of tea, in particular of green tea; anthocyans; extracts of rosemary; phenol acids, in particular chlorogenic acid; stilbenes, in particular resveratrol; sulphur-containing amino acid derivatives, in particular S-carboxymethylcysteine; ergothioneine;-N-acetyl-cysteine; chelating agents such as N,N′-bis(3,4,5-tri-methoxzybenzyl)ethylenediamine or one of its salts, metal complexes or esters; carotenoids such as crocetin; and various raw materials such as the mixture of arginine, histidine ribonucleate, mannitol, adenosine triphosphate, pyridoxine, phenylalanine, tyrosine and hydrolysed RNA sold by the Laboratoires Sérobiologiques under the commercial name CPP LS 2633-12F®, the water-soluble fraction of corn sold by the company SOLABIA under the commercial name Phytovityl®, the mixture of extract of fumitory and of extract of lemon sold under the name Unicotrozon C-49® by the company Induchem, and the mixture of extracts of ginseng, of apple, of peach, of wheat and of barley sold by the company PROVITAL under the commercial name Pronalen Bioprotect®.

As agents for trapping monocyclic or polycyclic aromatic compounds, which can be used in the composition according to the invention, mention may in particular be made of tannins such as ellagic acid; indole derivatives, in particular 3-indolecarbinol; extracts of tea, in particular of green tea, extracts of water hyacinth or Eichhornia crassipes; and the water-soluble fraction of corn sold by the company SOLABIA under the commercial name Phytovityl®.

Finally, as heavy-metal-trapping agents which can be used in the composition according to the invention, mention may in particular be made of chelating agents such as EDTA, the pentasodium salt of ethylenediaminetetramethylenephosphonic acid, and N,N′-bis(3,4,5-trimethoxybenzyl)ethylenediamine or one of its salts, metal complexes or esters; phytic acid; chitosan derivatives; extracts of tea, in particular of green tea; tannins such as ellagic acid; sulphur-containing amino acids such as cysteine; extracts of water hyacinth (Eichhornia crassipes); and the water-soluble fraction of corn sold by the company SOLABIA under the commercial name Phytovityl®.

The free-radical scavengers which can be used in the composition according to the invention comprise, besides certain antipollution agents mentioned above, vitamin E and its derivatives such as tocopheryl acetate; bioflavonoids; coenzyme Q10 or ubiquinone; certain enzymes such as catalase, superoxide dismutase, lactoperoxidase, glutathione peroxidase and quinone reductases; glutathione; benzylidenecamphor; benzylcyclanones; substituted naphthalenones; pidolates; phytanetriol; gamma-oryzanol; lignans; and melatonin.

According to a preferred embodiment of the invention, the composition used according to the process which is the subject of the invention contains, as additional active agent, a manganese salt, in particular manganese gluconate.

As indicated above, the composition according to the invention may also contain UVA and/or UBV screening agents, in the form of organic or inorganic compounds, the latter optionally being coated to make them hydrophobic.

The organic screening agents which are more particularly preferred are chosen from the following compounds (referred to according to CTFA nomenclature or under their chemical name): Ethylhexyl Salicylate, Ethylhexyl Methoxycinnamate, Octocrylene, Phenylbenzimidazole Sulphonic Acid, Benzophenone-3, Benzophenone-4. Benzophenone-5, 4-Methylbenzylidene-camphor, Terephthalylidene Dicamphor Sulphonic Acid, Disodium Phenyl Dibenzimidazole Tetrasulphonate, 2,4,6-tris(diisobutyl 4′-aminobenzalmalonate)-s-triazine, Anisotriazine, Ethylhexyltriazone, Diethyl-hexyl Butamido Triazone, Methylene Bis-Benzotriazolyl Tetramethylbutylphenol, Drometrizole Trisiloxane, 1,1-dicarboxy(2,2′dimethylpropyl)-4,4-diphenyl-butadiene, and mixtures thereof.

The inorganic screening agents preferably consist of zinc oxide, iron oxide, zirconium oxide, cerium oxide and/or titanium oxide. (amorphous or crystallized in rutile and/or anatase form), preferably of nanometric size (mean size of the primary particles: generally between 5 nm and 100 nm, preferably between 10 nm and 50 nm), optionally coated with alumina and/or stearic acid.

The invention will now be illustrated with the following nonlimiting examples.

### EXAMPLES

### Example 1

### Demonstration of the Relaxing Effect of Extracts of Boswellia serrata

Three extracts of Boswellia serrata were tested on a nerve-muscle coculture model which makes it possible to recreate a motor arc by innovating human striated muscle cells with explants of spinal cord and of spinal ganglia from rat embryos.

They were the extracts sold under the commercial names Soothex® by the company QUEST, Boswellin® by the company SABINSA and R3® by the company ZANDU.

This test predicts an anti-wrinkle effect, as was demonstrated by the applicant in the case of diazepam, which inhibited muscle fibre contractions in this model, and the anti-wrinkle activity of which was demonstrated in vivo.

a) Protocol

Human muscle cells derived from human muscle samples from a healthy donor are seeded in 15 mm-diameter wells (24-well culture dishes). After culturing for 10 days, these cells form a monolayer and fuse. At this stage, spinal cord explants from 13-day-old rat embryos, containing the spinal ganglion, are deposited onto the culture.

The first muscle fibre contractions are observed after coculturing for one week. After coculturing for 3 weeks, the muscle fibres are striated and possess mature differentiated neuromuscular junctions.

A muscle fibre having regular contractions (at least 60 contractions per minute) is then selected in three different culture wells and the number of contractions is counted over 30 seconds using image analysis software. The extracts tested, diluted in ethanol, are then incubated for 60 seconds in these wells, at the concentrations C₁ and C₂ of 0.005% and 0.01%. At the end of the incubation, the number of contractions is again counted over 30 seconds.

The percentage of inhibited contractions is then determined. The results are given in Table 1 below:

TABLE 1
Dermo-relaxing effect of the compounds
according to the invention

% inhibition of contractions

| Extract | C₁ = 0.005% | C₂ = 0.01% |
|---|---|---|
| Soothex ® (QUEST) | 26.46% | 37.08% |
| Boswellin ® (SABINSA) | 72.14% | — |
| R3 ® (ZANDU) | 0.67% | 38.44% |

This test thus demonstrates that the extracts of Boswellia serrata have a muscle-relaxing or decontracting effect which can be used to prepare cosmetic compositions with an anti-wrinkle effect, in particular for smoothing out expression lines.

### Example 2

### Demonstration of the Relaxing Effect of 3-O-acetyl-11-ketoboswellic Acid

The four pentacyclic triterpene acids contained, according to the literature, in extract of Boswellia serrata, namely beta-boswellic acid, 3-O-acetylboswellic acid, 11-ketoboswellic acid and 3-O-acetyl-11-ketoboswellic acid, were tested in a model of calcium flux in order to evaluate their capacity for inhibiting calcium channels and therefore their ability to relax lines and smooth out expression lines.

This test demonstrated the relaxing effect of 3-O-acetyl-11-ketoboswellic acid, which is clearly greater than that obtained for the other three acids tested.

This effect was confirmed in a test identical to that presented in Example 1, which showed a contraction-inhibiting effect of 74.7% at 5 μM and of 87% at 10 μM for this compound.

### Example 3

### Cosmetic Composition

This composition is prepared in a manner which is conventional for those skilled in the art. The amounts given in this example are indicated as weight percentages.

| | |
|---|---|
| Extract of Boswellia serrata | 3.00% |
| (Soothex ® from QUEST) | |
| Oils | 18.00% |
| Disodium EDTA | 0.1% |
| Mixed silicate (Laponite XLG from ROCKWOOD) | 1.0% |
| Triethanolamine | 0.5% |
| UV screening agents | 2.0% |
| Fillers | 4.0% |
| Glycerol | 7.0% |
| Stearic acid | 3.0% |
| Mixture of glyceryl stearate and of | 2.0% |
| polyethylene glycol stearate (100 OE) | |
| Polyethylene glycol stearate (20 OE) | 0.80% |
| Fatty alcohols | 1.0% |
| Preserving agents | 0.60% |
| Water | qs 100% |

This cream is intended to be applied to the face and the forehead in order to relax the face.

The above written description of the invention provides a manner and process of making and using it such that any person skilled in this art is enabled to make and use the same, this enablement being provided in particular for the subject matter of the appended claims, which make up a part of the original description, and including the use of 3-O-acetyl-11-ketoboswellic acid, or of a plant extract containing it, in a composition suitable for topical application to the skin, as an agent intended to soften lines and/or relax the skin. Moreover, the present specification enables the use of 3-O-acetyl-11-ketoboswellic acid, or of a composition, plant extract, etc. comprising it, to soften lines and/or relax the skin. In a preferred embodiment, the phrase “to soften lines” means “relaxing features,” as opposed to smoothing lines. The term “relax the skin” includes “relaxing features.”

As used above, the phrases “selected from the group consisting of,” “chosen from,” and the like include mixtures of the specified materials.

All references, patents, applications, tests, standards,.documents, publications, brochures, texts, articles, etc. mentioned herein are incorporated herein by reference. Where a numerical limit or range is stated, all values and subranges therewithin are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A method for relaxing the skin, comprising applying to the skin of a person in need thereof a skin relaxing effective amount of 3-O-acetyl-11-ketoboswellic acid.

2. The method according to claim 1, wherein said 3-O-acetyl-11-ketoboswellic acid is applied in the form of a plant extract comprising 3-O-acetyl-11-ketoboswellic acid.

3. The method according to claim 2, wherein said plant extract is selected from the group consisting of genus Boswellia plant extracts, genus Commiphora plant extracts, and mixtures thereof.

4. The method according to claim 2, wherein said plant extract is selected from the group consisting of Commiphora myrrha, Commiphora mukul, Commiphora opobalsamum, Commiphora playfairii, Commiphora abyssincia, Commiphora simplicifolia plant extracts, and mixtures thereof.

5. The method according to claim 2, wherein the plant extract is an extract of Commiphora mukul.

6. The method according to claim 2, wherein the plant extract is selected from the group consisting of Boswellia serrata, Boswellia carteri, Boswellia papyrifera, Boswellia frereana, Boswellia thurifera, Boswellia glabra, Boswellia oblongata, Boswellia socotrana extracts, and mixtures thereof.

7. The method according to claim 2, wherein the plant extract is an extract of Boswellia serrata.

8. The method according to claim 2, wherein the extract is an alcoholic extract obtained by extraction of a resinous exudate of Boswellia serrata with an alcoholic solvent.

9. The method according to claim 8, wherein the alcoholic extract is treated with alkali prior to application to the skin.

10. The method according to claim 9, wherein alkali treatment is followed by extraction of the alkali-treated alcoholic extract with an organic solvent to produce an aqueous phase and an organic phase prior to application to the skin.

11. The method according to claim 10, wherein the extraction is followed by acid treatment of the aqueous phase obtained prior to application to the skin.

12. The method according to claim 1, wherein said 3-O-acetyl-11-ketoboswellic acid is applied in the form of a composition comprising 3-O-acetyl-11-ketoboswellic acid, and wherein said composition comprises 0.001% to 0.1% by weight of 3-O-acetyl-11-ketoboswellic acid relative to the total weight of the composition.

13. The method according to claim 12, wherein the composition further comprises a manganese salt.

14. The method according to claim 13, wherein the manganese salt is manganese gluconate.

15. The method according to claim 1, wherein the 3-O-acetyl-11-ketoboswellic acid is applied to nasogenic furrows, lines between the eyebrows and/or lines on the forehead.

16. The method according to claim 2, wherein the plant extract is applied to nasogenic furrows, lines between the eyebrows and/or lines on the forehead.

17. The method according to claim 3, wherein the plant extract is applied to nasogenic furrows, lines between the eyebrows and/or lines on the forehead.

18. The method according to claim 11, wherein the plant extract is applied to nasogenic furrows, lines between the eyebrows and/or lines on the forehead.

19. The method according to claim 12, wherein the composition is applied to nasogenic furrows, lines between the eyebrows and/or lines on the forehead.

20. The method according to claim 14, wherein the composition is applied to nasogenic furrows, lines between the eyebrows and/or lines on the forehead.
